(19) United States
(12) Patent Application Publication
(10) Pub. No.: US 2019/0298212 A1
(43) Pub. Date: Oct. 3, 2019

(54) METHODS AND DEVICES FOR DETERMINING A SYNTHETIC SIGNAL OF BIOELECTRICAL ACTIVITY

(71) Applicant: DREEM, Paris (FR)

(72) Inventors: Quentin SOULET DE BRUGIÈRE, Pyla Sur Mer (FR); Hugo MERCIER, Chilly Mazarin (FR); Valentin THOREY, Paris (FR); Mathieu GALTIER, Paris (FR); Jérôme KALIFA, Paris (FR)

(21) Appl. No.: 16/465,837

(22) PCT Filed: Dec. 5, 2017
(86) PCT No.: PCT/FR2017/053383

§ 371 Date: May 31, 2019

(30) Foreign Application Priority Data
Dec. 5, 2016 (FR) 1661958

(51) Int. Cl. A61B 5/0478; A61B 5/00; A61B 5/048; A61B 5/04

(52) U.S. Cl. CPC A61B 5/0478; A61B 5/7221; A61B 5/0006; A61B 5/048; A61B 5/04012; A61B 5/7278

(57) ABSTRACT

Disclosed is a method and device for determining a robust synthetic signal indicative of a bioelectrical activity of an individual. Two measurement signals representative of physiological electrical signals of an individual are continuously acquired by electrodes. Two time series of confidence indices associated with the measurement signals are constructed. A synthetic signal is determined from the measurement signals and from the time series of confidence indices.

### FIELD OF THE DISCLOSURE

The present invention relates to methods and devices for determining a robust synthetic signal indicative of bioelectrical activity of an individual, in particular indicative of electroencephalographic activity.

Such methods and systems can in particular be used to determine a robust electroencephalographic (EEG) signal from an EEG acquisition device comprising, for example, dry electrodes.

The invention is of particular use in the field of acquisition and real-time processing of bioelectrical signals, in particular by portable autonomous devices for signal acquisition and processing.

### BACKGROUND

A major challenge in acquisition when using a portable device to measure bioelectrical signals, in particular from dry electrodes, is the robustness of the acquisition. It is a regular occurrence for one or more acquisition channels to become disconnected or not provide a signal of optimum quality. The quality of the acquisition can then vary greatly according to the state of the contact between electrode and skin.

### SUMMARY OF THE DISCLOSURE

The present invention is intended to overcome these disadvantages.

The present invention describes methods for obtaining a robust signal corresponding to bioelectrical activity, for example brain activity, by recombining signals from electrodes located close to one another.

The invention is based on the fact that spatially close electrodes capture similar bioelectrical signals. In particular, the rhythms, the characteristic patterns observed in bioelectrical signals occur at the same time within a limited spatial area.

For this purpose, a first object of the invention relates to a method for determining a robust synthetic signal indicative of bioelectrical activity of an individual, wherein:

at least two measurement signals respectively representative of two physiological electrical signals of an individual are acquired continuously, in particular by at least two electrodes placed in contact with or in proximity to an individual,

at least two time series of confidence indices respectively associated with the measurement signals are continuously constructed from said measurement signals,

a synthetic signal is continuously determined from said measurement signals and from said time series of confidence indices.

In preferred embodiments of the invention, one or more of the following may also be used:
each measurement signal is obtained from a differential measurement between two measurement electrodes, between a measurement electrode and a bias electrode, or between a bias electrode and an internal ground of a measurement signals processing unit;
the time series of confidence indices are constructed as a function of at least one among the power of the measurement signal, the power of the derivative of the measurement signal, the powers of the frequency bands of the measurement signal, a zero-crossing rate of the measurement signal, a detection of saturation of the measurement signal;
in order to determine the synthetic signal, a time series of synthetic signal values is determined such that, at each instant of the time series, the synthetic signal value corresponds to the value of the measurement signal associated with the highest confidence index for said instant;
in order to determine the synthetic signal, a time series of synthetic signal values is determined such that, at each instant of the time series, the synthetic signal value corresponds to a weighted average of the measurement signal values, each of the measurement signal values being respectively weighted according to the confidence index associated with the measurement signal for said instant;
in order to continuously determine the synthetic signal, each of said measurement signals is continuously analyzed in order to identify predefined patterns of bioelectrical activity and each of said measurement signals is associated with a list of identified patterns by analyzing said measurement signal, each identified pattern being associated with a recording time instant or time window in the associated measurement signal;
the synthetic signal comprises a list of predefined patterns of bioelectrical activity associated with acquisition time instants or time windows;
in order to determine the synthetic signal, the list of patterns constituting the synthetic signal is constructed from lists of patterns associated with each measurement signal, each pattern of each of said lists of patterns being weighted by the confidence index associated with the associated measurement signal for the recording time instant or time window associated with said pattern;
a virtual reference is determined from a measurement of the potential of a measurement electrode or bias electrode and as a function of a measurement of a current exchanged between a bias electrode and the individual.

The invention also relates to a device for acquiring a robust synthetic signal indicative of bioelectrical activity of an individual, comprising:

at least two electrodes placed in contact with, or in proximity to, an individual in order to continuously acquire at least two measurement signals respectively representative of two physiological electrical signals,

a processing unit comprising

inputs for receiving the measurement signals from the electrodes, and

processing arranged for

continuously constructing, from the measurement signals, at least two time series of confidence indices respectively associated with said measurement signals,

continuously determining a synthetic signal based on said measurement signals and said time series of confidence indices.

### BRIEF DESCRIPTION OF THE DRAWINGS

Other features and advantages of the invention will become apparent from the following description of one of its embodiments, given as a non-limiting example, with reference to the accompanying drawings.

In the drawings:

FIG. 1 and FIG. 2 are schematic views of a device according to one embodiment of the invention,

FIG. 3 is a flowchart illustrating a first embodiment of a method according to one embodiment of the invention,

FIG. 4 is a flowchart illustrating a second embodiment of a method according to one embodiment of the invention.

In the different figures, the same references designate identical or similar elements.

### DETAILED DESCRIPTION OF THE EMBODIMENTS

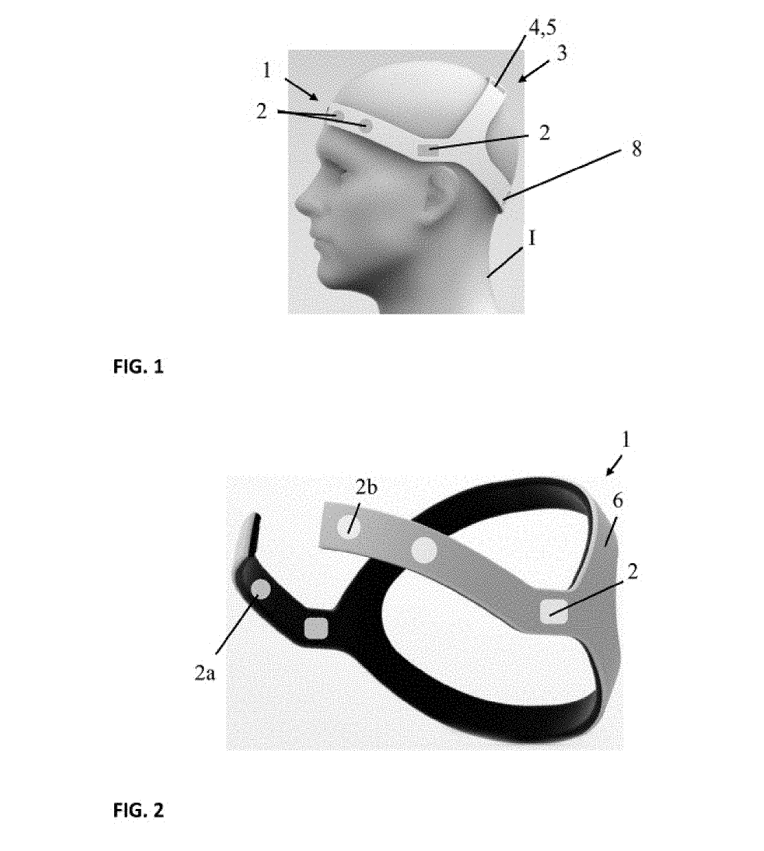

FIG. 1 illustrates a device 1 for acquiring a robust synthetic signal indicative of bioelectrical activity of an individual I according to one embodiment of the invention.

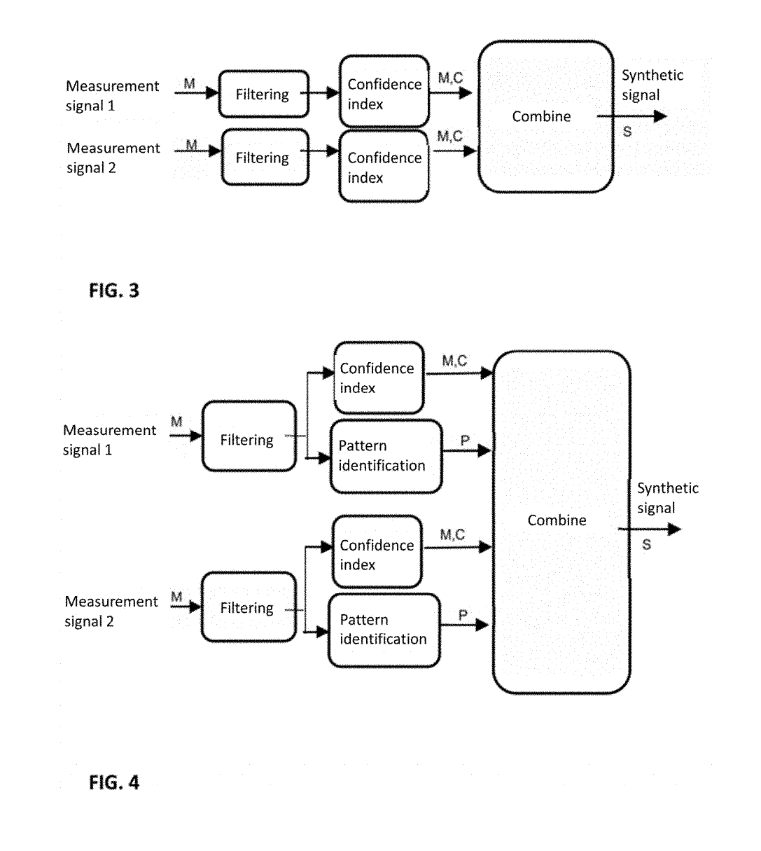

Such a device 1 is able to implement a method for determining a robust synthetic signal indicative of bioelectrical activity of an individual according to the invention, for example as illustrated in FIG. 3.

As can be seen in FIG. 1, the device 1 comprises at least two electrodes 2. The electrodes are placed in contact with, or in proximity to, the individual I.

The electrodes 2 comprise measurement electrodes 2b which enable the continuous acquisition of at least two measurement signals M respectively representative of two physiological electrical signals in the individual I.

The electrodes may comprise a bias electrode 2b adapted to maintain a predefined potential at the head relative to a ground of the device 1.

The measurement signals are determined from measurements of the potentials of the electrodes 2 and in particular are determined by measuring a potential difference between two electrodes.

“Potential” is understood to mean a signal from a single electrode, and “lead” (for example as detailed below) to mean the difference of two potentials.

A measurement signal is for example:
a lead between measurement electrodes that are relatively far apart, for example Mastoid/Forehead,
a lead between adjacent or close measurement electrodes (Forehead-Forehead, Hair-Hair, Forehead-Hair),
a lead between an electrode (for example Forehead) and a bias electrode. The signal of a bias electrode is similar to that of a measurement electrode, so their characteristics can be exploited. Moreover, in the case of detachment of a measurement electrode, for example left forehead (Fp1) (respectively right forehead (Fp2)), if the Fp2-bias (respectively Fp1-bias) lead is measured then the latter signal can be used to characterize the physiological electrical signals.
a lead between two measurement and/or bias electrodes, modified by an analog operation (for example filtering or modulation), or modulated by measuring the current output from the bias towards the electronic circuit, to obtain a virtual reference,
a signal resulting from combining several leads, as indicated above, for example by means of an analog operation (such as filtering, modulation, averaging, etc.) allowing for example establishing a virtual reference, and/or from combining with a third signal used to analyze a physiological activity (for example sleep) and events characteristic of said physiological activity.

For example, it is possible to modulate the potential coming from a measurement or bias electrode as a function of the current output from the bias towards the electronic circuit. This allows for example correcting the potential coming from an electrode so that it corresponds to a virtual reference.

“Virtual reference” is understood to mean a potential that is similar to a signal measured at a point in the human body distant from or shielded from brain waves, for example a signal similar to a signal coming from an electrode placed on a mastoid or earlobe.

A virtual reference may be determined from a potential measured at a point, of the human body or of the electronic circuit of the device, which is different from a conventional reference for the measurement of bioelectrical signals (for example electroencephalogram) such as the mastoid, earlobe, or nose. The raw potential measured at such a point is thus corrected to approximate the potential expected for a reference, in other words so as to be independent from the brain waves to the extent possible. Measurement at such a point is advantageously easier to access or more comfortable (for example the forehead) than measurement at a conventionally used reference point such as the earlobe.

It is then possible to obtain a measurement signal based on a measurement electrode and the virtual reference.

The measurement signals can come from leads that may or may not have electrodes in common.

For example, three measurement signals can be acquired which respectively correspond to the following leads:
left forehead-right forehead
left temple-left forehead
right temple-right forehead.

The device 1 also comprises a processing unit 3 comprising means 4 for receiving the measurement signals of the electrodes 2, and processing means 5.

The processing means 5 may for example comprise one or more processors as well as one or more appropriate memories.

The receiving means 4 may for example comprise connectors as well as electronics for communicating with and/or controlling the electrodes.

The processing means 5 and the receiving means 4 may be integrated into a single chip.

The device may comprise a single frame 6 on which are mounted the electrodes 2, the processing unit 3, the receiving means 4, and the processing means 5. The device 1 may also comprise a battery. The battery 8 may be mounted on the frame 6.

The processing means 5 are adapted for
continuously constructing, from the measurement signals M, at least two time series of confidence indices C respectively associated with said measurement signals M,
continuously determining a synthetic signal S from said measurement signals and from said time series of confidence indices, as will now be described in more detail.

Reference will now be made more particularly to FIGS. 3 and 4 which describe various methods and embodiments of the invention.

In a first embodiment of the invention, illustrated in FIG. 3, the synthetic signal comprises measurement signal values, weighted where appropriate, as detailed below.

In a second embodiment of the invention, illustrated in FIG. 4, the synthetic signal comprises a list of predefined patterns of bioelectrical activity associated with acquisition time instants or time windows.

Such predefined patterns of bioelectrical activity are for example slow oscillations, sigma waves (“spindles”), K-complexes, slow rhythms, micro-awakenings, alpha rhythms, etc.

We first refer to FIG. 3 which illustrates the first embodiment of the invention. In this embodiment, the synthetic signal includes values, weighted where appropriate, of several physiological electrical signals that are close in order to obtain a signal of good quality.

“Physiological electrical signals that are close” is understood to mean for example measurement signals coming from electrodes placed in positions that are close, for example two electrodes placed in forehead positions. Close electrodes are for example less than 10 centimeters apart, for example less than 5 centimeters.

“Signal of good quality” is understood to mean the detection and discarding of poor quality signals and/or the possible combining of similar signals of good quality. The quality of the signal may in particular be quantified as described below.

A first step of the method may include filtering steps, for example filtering of 50 Hz or 60 Hz, frequencies above 200 Hz, and possibly other bands. Different filtering methods are possible, for example Butterworth filtering, wavelet or Kalman filtering.

The method for quantifying the signal quality is adapted to allow recognizing signal amplitudes or abnormal waveforms (i.e. not corresponding for example to the encephalographic signal usually observed).

For this purpose, a time series of confidence indices associated with said measurement signal is continuously constructed for each measurement signal.

“Continuously constructed” is understood to mean that the confidence indicator series are constructed in real time (for example in soft real time), simultaneously and in parallel with the acquisition of said measurement signals.

“Continuously” is understood to mean that the operations and/or steps are repeated over time during a period of measurement signal acquisition by the device, in particular repeated periodically or quasi-periodically.

Each confidence index can be calculated from a quantity derived from the measurement signal, for example selected from the power of the measurement signal, the power of the derivative of the measurement signal, the different frequency bands of the measurement signal, the value of the impedance of the measurement signal, the zero-crossings rate of the measurement signal, detection of saturation of an amplifier of the measurement signal acquisition chain, the values of the measurement signal.

In one embodiment, at least one additional measurement signal representative of a physiological activity of the individual may be acquired. The additional measurement signal may for example be measured by an accelerometer.

The confidence index of a measurement signal can also be indicative of confidence in the data of other measurement signals. For example, the measurement signal obtained by an accelerometer, indicative of the movements of the user, can be used to determine an index of confidence in the values measured by electrodes.

In an exemplary embodiment given as an illustration, the device can exploit the presence of the frequency of the electrical supply system, 50 Hz, to detect the detachment of an electrode and eliminate a signal. Thus, when measuring a lead between two electrodes in contact with the user or in proximity for proximity electrodes, for example FP1-FP2, the 50 Hz frequencies present in the two measurement signals largely compensate for each other. When one of the electrodes comes off, or moves away in the case of proximity electrodes, the power of the signal in the 50 Hz band increases significantly for said electrode. The signal derived from FP1-FP2 then comprises a very high 50 Hz power. This makes it possible to determine a signal confidence index.

Indices and/or quantities derived from the measurement signal can be calculated for time windows of variable duration, depending on the indicator concerned.

Indices may have binary values (for example, a boolean that indicates whether or not the signal is of good quality), categorical values, or values continuously varying over a predefined range (for example quantifying the probability of having a signal of good quality).

The operation of constructing a confidence index from one or more measurement signals may comprise determining one or more quantities indicated above and then comparing them with one or more predefined thresholds, for example starting from the values usually encountered in electro-encephalography.

The operation of constructing a confidence index may also employ a predictor obtained by a learning method (for example a random forest or a neural network) and trained on a learning database constructed from clips of measurement signals identified as being of good or bad quality. This learning database may include index values (for example those presented above) calculated on these same identified signals.

Once a confidence index is determined, the method includes continuously determining a synthetic signal from the measurement signals and from the time series of confidence indices.

This makes it possible to discard poor quality signals in real time. Several alternative embodiments are possible for constructing the synthetic signal from the signals at a given moment.

According to a first variant, the synthetic signal at a given moment is obtained from the measurement signal having the best quality at said moment. If the quality decreases below a threshold at a later moment, the synthetic signal is determined from the measurement signal having the best quality at said later moment.

According to a second variant, the synthetic signal at a given moment is obtained by combining the measurement signals at said moment which are of sufficient quality as indicated by the confidence index. In this manner, it is possible to average the measurement signals acquired at positions that are close on the individual in order to increase the robustness of the resulting synthetic signal.

The synthetic signal is then determined by averaging the measurement signals acquired at close positions and having sufficient quality as indicated by the confidence index. The average may be weighted by the confidence index of each measurement signal at the associated moment.

Other strategies for combining measurement signals are of course conceivable.

Moreover, as indicated above, the confidence index associated with a measurement signal at a given moment can be determined from another measurement signal, for example an additional measurement signal representative of a physiological activity of the individual such as the one provided by an accelerometer.

We will now refer to FIG. 4 which illustrates the second embodiment of the invention. In this embodiment, the synthetic signal comprises a list of predefined patterns of bioelectrical activity associated with acquisition time instants or time windows.

This second embodiment of the invention may be combined with the first embodiment, for example by implementing it in parallel with the first embodiment described above.

This second embodiment makes it possible to directly obtain events of interest identified in the measurement signals, by performing the detections and analyses on each measurement signal before combining the detections according to the confidence indices.

In this embodiment, a confidence index is calculated once again as indicated above.

Then, or in parallel, the measurement signal is processed in order to identify predefined patterns of bioelectrical activity. Each of the measurement signals is then associated with a list of identified patterns, each identified pattern being associated with a recording time instant or time window in the associated measurement signal.

The list of identified patterns comprises for example patterns associated with slow oscillations, sigma waves (“spindles”), K-complexes, slow rhythms, micro-awakenings, alpha rhythms, etc.

For each acquisition channel, i.e. each measurement signal, having a decent quality as indicated by the confidence index, we then define a probability that each of these events has taken place. These probabilities are then compared to determine the presence of one of the events associated with the patterns.

In particular, for each recording time instant or time window of a measurement signal, and for each pattern in the list of identified patterns, a probability is determined that the event associated with the pattern has taken place, in other words that the bioelectrical activity associated with the pattern has occurred during the time instant or time window concerned.

The synthetic signal is then determined by constructing a list of the patterns that constitute the synthetic signal, from the lists of patterns associated with each measurement signal. Each pattern of each of the lists of patterns associated with the measurement signals is advantageously weighted by the confidence index associated with the associated measurement signal for the recording time instant or time window associated with said pattern.

The step of identifying the predefined patterns of bioelectrical activity may be implemented based on one or more indicators calculated on the signals. The indicators may be chosen, for example, from the power of the measurement signal, the power of the derivative of the measurement signal, the different frequency bands of the measurement signal, the value of the impedance of the measurement signal, the zero-crossing rate of the measurement signal, the detection of saturation of an amplifier of the measurement signal acquisition chain, the values of the measurement signal. The indicators may be calculated on time windows of variable duration, depending on the indicator concerned.

The operation of identifying the predefined patterns of bioelectrical activity may comprise determining one or more of the indicators indicated above and then comparing them with one or more predefined threshold(s), for example based on the values usually encountered in electro-encephalography.

The operation of identifying the predefined patterns of bioelectrical activity may also employ a predictor obtained by a learning method (for example a random forest or a neural network) and trained on a learning database constructed from measurement signal clips with identified patterns. This learning database may include indicator values (for example those presented above) calculated on these same identified signals.

In particular, the operation of identifying the predefined patterns can take into account the fact that the event associated with a pattern has a high probability for several acquisition channels at the same time instant or during the same time window of the recording.

In addition, the identification operation may favor a specific acquisition channel for identifying a pattern, the event associated with the pattern being more likely to be identified by this acquisition channel than in the other acquisition channels.

Finally, the synthetic signal is continuously determined from the measurement signals and from the time series of confidence indices. This step will use the previously calculated confidence index as well as the lists of patterns identified in the measurement signals.

The probabilities of the events associated with the previously calculated patterns in each of the signals are, for example, weighted by the quality of these signals as determined from the confidence index associated with the corresponding time instant.

An accordingly weighted average of the pattern detections makes it possible to reinforce the robustness of the pattern detections by taking into account the different measurement channels. Here again, the signals having a confidence index lower than a given threshold can be excluded from the average and the determination of the synthetic signal can take into account only those signals having a confidence index above a fixed threshold.

In one particular embodiment of the invention, it is possible to average the measurement signals coming from several reference electrodes (for example from two mastoids) in order to obtain a super-reference replacing said references for the signal acquisition.

Weighting the average over time, which makes it possible to calculate the super-reference, can also be done as a function of the confidence index determined by analysis of the signals from the reference electrodes, as detailed above.

1. Method for determining a robust synthetic signal indicative of bioelectrical activity of an individual, wherein:
at least two measurement signals respectively representative of two physiological electrical signals of an individual are acquired continuously, in particular by at least two electrodes placed in contact with or in proximity to an individual,
at least two time series of confidence indices respectively associated with the measurement signals are continuously constructed from said measurement signals,
a synthetic signal is continuously determined from said measurement signals and from said time series of confidence indices.

2. Method according to claim 1, wherein each measurement signal is obtained from a differential measurement between two measurement electrodes, between a measurement electrode and a bias electrode, or between a bias electrode and an internal ground of a measurement signals processing unit.

3. Method according to claim 1, wherein the time series of confidence indices are constructed as a function of at least one among the power of the measurement signal, the power of the derivative of the measurement signal, the powers of the frequency bands of the measurement signal, a zero-crossing rate of the measurement signal, a detection of saturation of the measurement signal.

4. Method according to claim 1, wherein, in order to determine the synthetic signal, a time series of synthetic signal values is determined such that, at each instant of the time series, the synthetic signal value corresponds to the value of the measurement signal associated with the highest confidence index for said instant.

5. Method according to claim 1, wherein, in order to determine the synthetic signal, a time series of synthetic signal values is determined such that, at each instant of the time series, the synthetic signal value corresponds to a weighted average of the values of the measurement signals, each of the measurement signal values being respectively weighted according to the confidence index associated with the measurement signal for said instant.

6. Method according to claim 1, wherein, in order to continuously determine the synthetic signal, each of said measurement signals is continuously analyzed in order to identify predefined patterns of bioelectrical activity and each of said measurement signals is associated with a list of identified patterns by analyzing said measurement signal, each identified pattern being associated with a recording time instant or time window in the associated measurement signal.

7. Method according to claim 6, wherein the synthetic signal comprises a list of predefined patterns of bioelectrical activity associated with acquisition time instants or time windows.

8. Method according to claim 7, wherein, in order to determine the synthetic signal, the list of patterns constituting the synthetic signal is constructed from lists of patterns associated with each measurement signal, each pattern of each of said lists of patterns being weighted by the confidence index associated with the associated measurement signal for the recording time instant or time window associated with said pattern.

9. Method according to claim 6, wherein, for each recording time instant or time window in the associated measurement signal, and for each pattern of the list of identified patterns, a probability of an event associated with the pattern taking place is determined.

10. Method according to claim 1, wherein a virtual reference is determined from a measurement of the potential of a measurement electrode or of a bias electrode and as a function of a measurement of a current exchanged between a bias electrode and the individual.

11. Device for acquiring a robust synthetic signal indicative of bioelectrical activity of an individual, comprising:
at least two electrodes placed in contact with, or in proximity to, an individual, in order to continuously acquire at least two measurement signals respectively representative of two physiological electrical signals,
a processing unit comprising
inputs for receiving the measurement signals from the electrodes, and
processing arranged for
continuously constructing, from the measurement signals, at least two time series of confidence indices respectively associated with said measurement signals,
continuously determining a synthetic signal based on said measurement signals and said time series of confidence indices.

12. Method according to claim 2, wherein the time series of confidence indices are constructed as a function of at least one among the power of the measurement signal, the power of the derivative of the measurement signal, the powers of the frequency bands of the measurement signal, a zero-crossing rate of the measurement signal, a detection of saturation of the measurement signal.

13. Method according to claim 2, wherein, in order to determine the synthetic signal, a time series of synthetic signal values is determined such that, at each instant of the time series, the synthetic signal value corresponds to the value of the measurement signal associated with the highest confidence index for said instant.

14. Method according to claim 3, wherein, in order to determine the synthetic signal, a time series of synthetic signal values is determined such that, at each instant of the time series, the synthetic signal value corresponds to the value of the measurement signal associated with the highest confidence index for said instant.

15. Method according to claim 2, wherein, in order to determine the synthetic signal, a time series of synthetic signal values is determined such that, at each instant of the time series, the synthetic signal value corresponds to a weighted average of the values of the measurement signals, each of the measurement signal values being respectively weighted according to the confidence index associated with the measurement signal for said instant.

16. Method according to claim 3, wherein, in order to determine the synthetic signal, a time series of synthetic signal values is determined such that, at each instant of the time series, the synthetic signal value corresponds to a weighted average of the values of the measurement signals, each of the measurement signal values being respectively weighted according to the confidence index associated with the measurement signal for said instant.

17. Method according to claim 4, wherein, in order to determine the synthetic signal, a time series of synthetic signal values is determined such that, at each instant of the time series, the synthetic signal value corresponds to a weighted average of the values of the measurement signals, each of the measurement signal values being respectively weighted according to the confidence index associated with the measurement signal for said instant.

18. Method according to claim 2, wherein, in order to continuously determine the synthetic signal, each of said measurement signals is continuously analyzed in order to identify predefined patterns of bioelectrical activity and each of said measurement signals is associated with a list of identified patterns by analyzing said measurement signal, each identified pattern being associated with a recording time instant or time window in the associated measurement signal.

19. Method according to claim 3, wherein, in order to continuously determine the synthetic signal, each of said measurement signals is continuously analyzed in order to identify predefined patterns of bioelectrical activity and each of said measurement signals is associated with a list of identified patterns by analyzing said measurement signal, each identified pattern being associated with a recording time instant or time window in the associated measurement signal.

20. Method according to claim 4, wherein, in order to continuously determine the synthetic signal, each of said measurement signals is continuously analyzed in order to identify predefined patterns of bioelectrical activity and each of said measurement signals is associated with a list of identified patterns by analyzing said measurement signal, each identified pattern being associated with a recording time instant or time window in the associated measurement signal.
